(19) United States
(12) Patent Application Publication
(10) Pub. No.: US 2018/0364223 A1
(43) Pub. Date: Dec. 20, 2018

(54) PORTABLE SINGLE-MOLECULE BIO-SENSING DEVICE

(71) Applicant: Krista Michelle Fretes, Flushing, NY (US)

(72) Inventor: Krista Michelle Fretes, Flushing, NY (US)

(21) Appl. No.: 15/624,751

(22) Filed: Jun. 16, 2017

(51) Int. Cl. G01N 33/543

(52) U.S. Cl. CPC G01N 33/54366; B01L 3/5027; G01N 33/5438

(57) ABSTRACT

A portable single-molecule bio-sensing device, comprising a substrate, a waveguide positioned on an upper surface of the substrate, a sampling channel edged into the waveguide, the sampling channel running a longitudinal length of the waveguide, a coupling channel edged into the waveguide, the coupling channel running perpendicular to the sampling channel, a pair of nanostructures secured to an outer surface of the coupling channel on opposite sides of the sampling channel, the nanostructures configured to form a gap that functions as a plasmon antenna, and a nanoaperture affixed to an outer surface of the sampling channel within the gap, an outer surface of the nanoaperture in direct contact with each of the pair of nanostructures and the nanoaperture including at least one aperture extending the full length of nanoaperture and in-line with the sampling channel.

### FIELD OF THE INVENTION

This disclosure relates generally to a portable single-molecule bio-sensing device.

### BACKGROUND

Generations of nucleic acid sequencing technologies, from Sanger sequencing to nanopore sequencing, have developed as a result of the important and essential application of genetic sequencing, the most essential of these being personalized medicine for a wide variety complex disease treatments including cancer treatment. Additional applications that make an efficient nucleic acid sequencing platform highly sought after include vaccine research, epidemic prevention, food monitoring, and forensic sample analysis.

Nanopore sequencing is one current technology that is well-known in the field of the nucleic acid sequencing. In nanopore sequencing, nucleic acid in a solution is run through an electric field and directed into either a biological or solid state nanopore embedded in a membrane substrate. Changes in ionic current running through the membrane are caused by nucleic acid bases translocating through the nanopore and altering the electric field surrounding the nanopore. These changes in ionic current may be detected and measured using sensitive current-measuring devices. Nanopore sequencing has allowed real-time sequencing without the need to label nucleic acid and advanced to between one and two kilobit read lengths.

While nanopore sequencing does exhibit the capability for reasonably long-read nucleic acid sequencing, problems have been found with this method that reduce its efficiency and ability to make personalized medicine, and other awaited applications, a reality. Specifically, while this sequencing method has achieved processing speeds of up to 250 bases per second, this speed is not sufficient for in the field forensic analysis. Also, while this sequencing method has exhibited a base reading accuracy between 92% and 98%, this accuracy range is still not sufficient for haplotyping sequencing and personalized medicine type applications.

The use of localized surface plasmon resonance (“LSPR”) is another developing technology in the field of the nucleic acid sequencing. LSPR is a phenomenon described by the interaction between electron oscillation on the surface of metal nanoparticles and the electric field of incident light. At a resonance wavelength, a localized surface plasmon polariton is excited at the nanoparticle surface, with the resonant frequency highly dependent on the size, geometry, and distance between the nanoparticles as well as the refractive index of the surrounding medium. The localized surface plasmon field is highly sensitive to changes in the refractive index of the surrounding medium, which can be the result of interactions with biomolecules surrounding the nanoparticle surface. Changes in the refractive index can be conveyed as shifts in the resonant wavelength of the light reflected by the plasmon field. Nanoparticles used in these applications are commonly of noble metal composition for their ability to excite LSPR in the visible light range.

In LSPR configurations, the geometry of the nanoparticles plays a large role in enhancing the electric field between two nanoparticles where LSPR occurs. Rod and triangle shaped nanoparticles significantly enhance the localized plasmon field between the edges of two closely neighboring nanoparticles. When using triangle shaped nanoparticles, this effect is more pronounced when the triangle shaped nanoparticles are arranged tip-to-tip, also known as a bowtie configuration and thereby creating a plasmon antenna. In this configuration, the tips of two triangular nanoparticles tightly confine an optical field, thereby enhancing it at values demonstrated to be larger than 10 to the second power for gap spaces smaller than 20 nanometers. This effect occurs with incident light that is polarized and whose electric field is parallel to the major axis of the bowtie configuration. The incident light creating a highly confined electric field within the gap region of the plasmon antenna in between the tips of the triangular nanoparticles positioned in a bowtie configuration. The result of this effect is a gap region with an enhanced sensitivity to small changes in the surrounding refractive index and thereby allowing for the detection of a single molecule within the local environment of the confined field within the plasmon antenna.

What is needed is a faster, cheaper, portable, and accessible devices for molecular detection that can be used by either experts in the fields or by general consumers. What is needed in view of these existing technologies is a portable device that provides for the direct measurements of shifts in light intensity and frequency correlating directly to specific molecular components translocating through a plasmon antenna.

### BRIEF SUMMARY

In an effort to address the above-described needs, the present application discloses an exemplary embodiment of a portable single-molecule bio-sensing device, comprising a substrate, a waveguide positioned on an upper surface of the substrate, a sampling channel edged into the waveguide, the sampling channel running a longitudinal length of the waveguide, a coupling channel edged into the waveguide, the coupling channel running perpendicular to the sampling channel, a pair of nanostructures secured to an outer surface of the coupling channel on opposite sides of the sampling channel, the pair of nanostructures configured to form a gap that functions as a plasmon antenna, and a nanoaperture affixed to an outer surface of the sampling channel within the gap, an outer surface of the nanoaperture in direct contact with each of the pair of nanostructures and the nanoaperture including at least one aperture extending the full length of nanoaperture and in-line with the sampling channel.

The present application further discloses an exemplary embodiment of a method of bio-sensing a single-molecule, the method comprising energizing a plasmon field on the surface of a waveguide, introducing a medium containing a nucleic acid, protein, molecules and viral/cell components to be sampled into a sampling channel etched into the waveguide, translocating the medium though a nanoaperture in direct contact with surrounding nanostructures forming a plasmon antenna, detecting shifts in light intensity and frequency produced by the interaction the nucleic acid or protein with the plasmon field as it is translocated through the plasmon antenna, and identifying the translocated nucleic acid or protein based on the detected shifts in light intensity and frequency.

The present application further discloses an exemplary embodiment of a portable single-molecule bio-sensing device, comprising a substrate, a waveguide positioned on an upper surface of the substrate, an additional layer of silicone dioxide (SiO₂) grown on an upper surface the waveguide, a pair of nanostructures embedded within the additional layer, the pair nanostructures configured to form a gap that functions as a plasmon antenna, a sampling channel edged into the additional layer, the sampling channel running a longitudinal length of the additional layer and positioned within the gap that functions as the plasmon antenna, and a nanoaperture affixed to the outer surface of the sampling channel, the nanoaperture positioned to be in direct contact with each of the pair of nanostructures and the nanoaperture including at least one aperture extending the full length of nanoaperture and in-line with the sampling channel.

### BRIEF DESCRIPTION OF THE DRAWINGS

This disclosure is further described in the detailed description that follows, with reference to the drawings, in which:

FIG. 1 is a diagram of a portable single-molecule bio-sensing device according to a disclosed embodiment.

FIG. 2A is a top down view of a plasmon antenna implemented within the portable single-molecule bio-sensing device according to a disclosed embodiment.

FIG. 2B is an inline view of the plasmon antenna implemented within the portable single-molecule bio-sensing device according to a disclosed embodiment.

FIG. 2C is an inline view of an alternate nanoaperture shape implemented within the portable single-molecule bio-sensing device according to a disclosed embodiment.

FIG. 3 is a block diagram showing a light source unit, a photodetector unit, a micropump, and a power source as implemented in conjunction with the portable single-molecule bio-sensing device according to a disclosed embodiment.

FIG. 4 is a diagram of a portable single-molecule bio-sensing device according to another embodiment.

### DETAILED DESCRIPTION

An exemplary embodiment of a portable single-molecule bio-sensing device is disclosed. As required, detailed embodiments of the disclosed device are disclosed herein however, it is to be understood that the disclosed embodiments are merely exemplary of the invention which may be embodied in various and alternative forms. The figures are not necessarily to scale and some features may be exaggerated or minimized to show details of particular components. Therefore, the structural and functional details disclosed herein are not to be interpreted as limiting, but merely as representative for teaching one of ordinary skill in the art to variously employ the present disclosure.

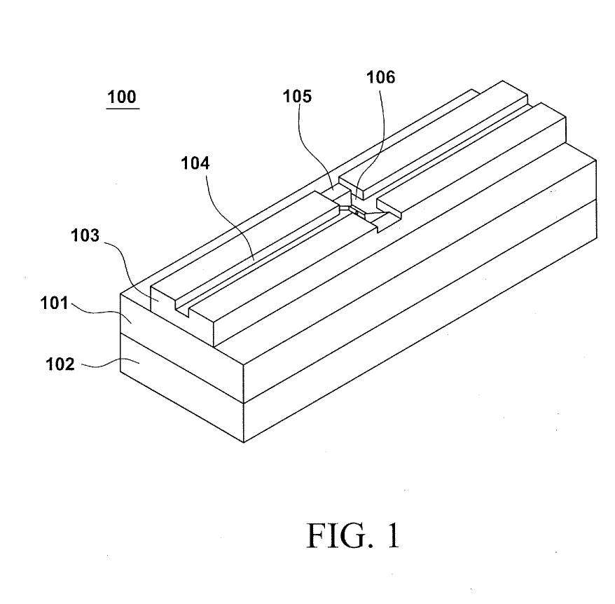

In FIG. 1, a diagram of a portable single-molecule bio-sensing device according to the disclosed embodiment is shown. As shown in FIG. 1, the portable single-molecule bio-sensing device 100 includes a waveguide 103, a top layer substrate 101, a lower layer substrate 102, a sampling channel 104, a coupling channel 105, and a plasmon antenna 106.

The waveguide 103 is a single-mode ridge waveguide composed of silicone nitrate (Si₃N₄) that has been deposited on a top layer substrate of silicone dioxide (SiO₂) 101. The waveguide 103 may be done using either low pressure vapor deposition or plasma enhanced vapor deposition. The waveguide 103 may also be formed using electron beam lithography, reactive ion etching, or any other reasonable method known to one of ordinary skill in the art. In the disclosed embodiment, the waveguide 103 has a width ranging between 400-900 nanometers and a height ranging between 200-300 nanometers.

The top layer substrate 101 of silicon dioxide (SiO₂) functions as a cladding layer to the waveguide 103. The silicone nitrate (Si₃N₄) layer and the silicon dioxide (SiO₂) layer have highly contrasting refractive indexes which helps minimize the amount of light that leaks out of the waveguide 103. The top layer substrate 101 of silicon nitrate cladding layer (SiO₂) is itself thermally grown on a lower layer substrate 102 of silicon (Si), the top layer substrate 101 thermally grown to a width of approximately between 2 to 5 micrometers in the disclosed embodiment.

The sampling channel 104 is edged into the waveguide 103 along the longitudinal length of the waveguide 103. The sampling channel 104 may be formed using electron beam lithography or any other reasonable method known to one or ordinary skill in the art.

While the disclosed embodiment discloses a ridged waveguide, any type of single-mode waveguide know to one of ordinary skill in the art may be used, including slot, strip-loaded, and plasmonic waveguide types.

The coupling channel 105 is edged into the waveguide 103 along the lateral length of the waveguide 103 and intersecting the sampling channel 104. The coupling channel 105 is formed in a similar manner as the sampling channel 104. The position of the coupling channel 105 may be adjusted as needed for specific implementations as to intersect the sampling channel 104 at any point along the longitudinal length of the waveguide 103.

An adhesion layer may be adsorbed onto the outer surfaces of the etched sampling channel 104 and the coupling channels 105 to further enable the adhesion of nanostructures comprising the plasmon antenna 106 onto the surfaces of these etched channels. To achieve maximum coupling between light traveling through the waveguide 103 and the plasmon antenna 106, the adhesion layer should be kept as thin as possible. In the disclosed embodiment, the adhesion layer has a thickness that is less than 5 nanometers.

The plasmon antenna 106 is positioned at the intersection of the sampling channel 104 and the coupling channel 105. A light source is coupled to and delivers light into one end of the waveguide 103. As a result of the delivered light, a transverse electric mode is excited on the outer surface of the sampling channel 104 and a plasmon field is generated within the plasmon antenna 106.

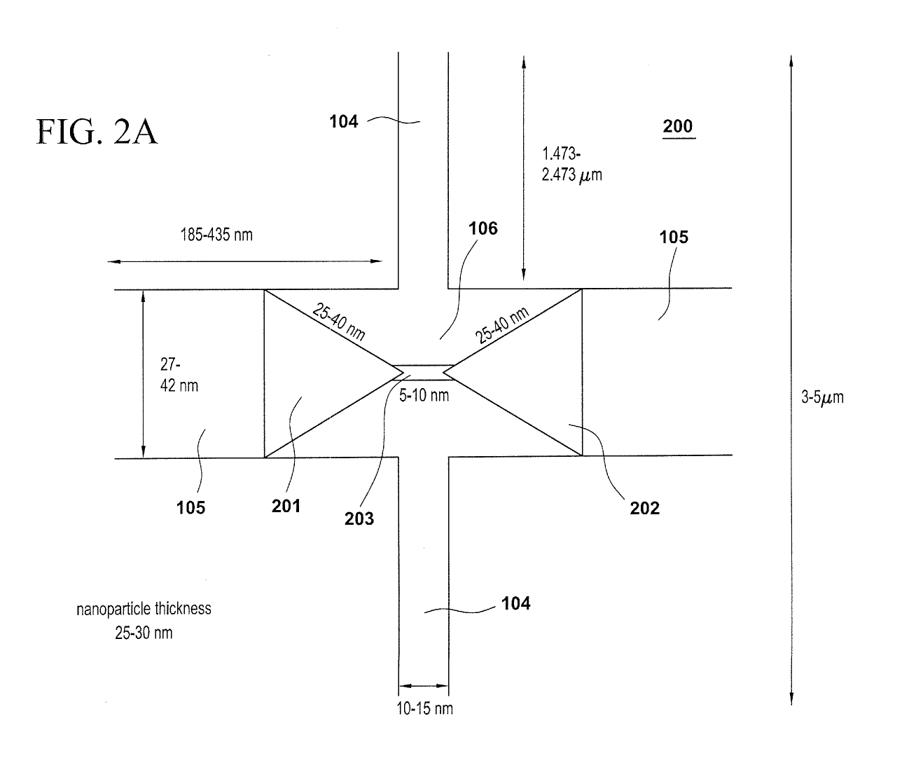

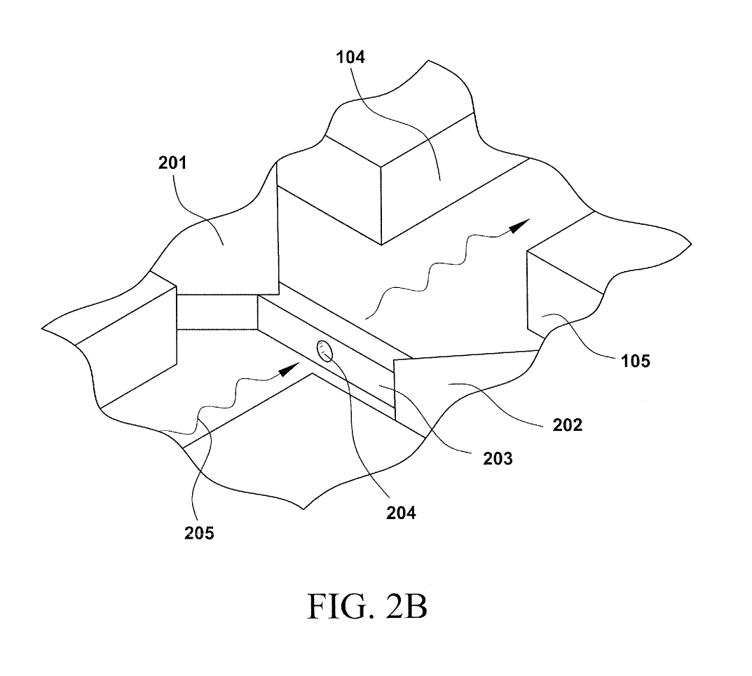

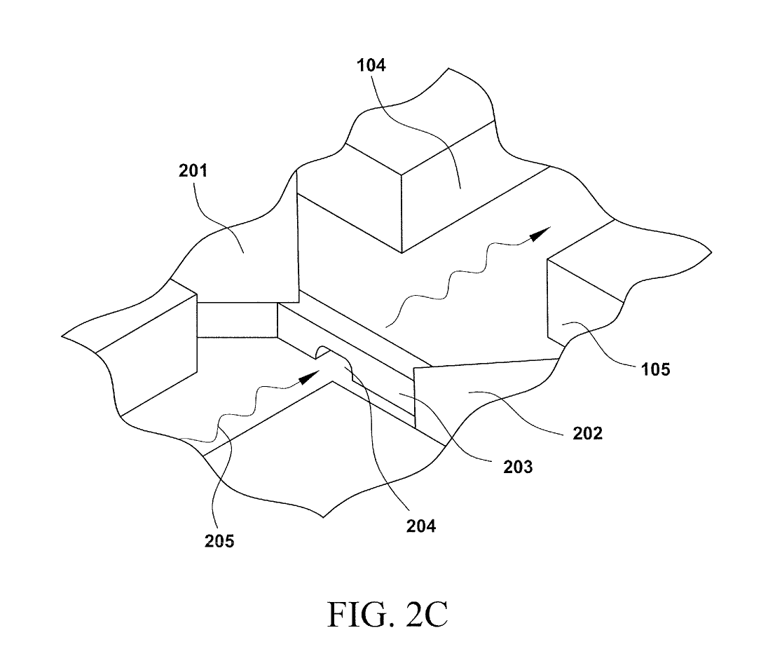

In FIG. 2A, a top down view of a plasmon antenna implemented within the portable single-molecule bio-sensing device according to the disclosed embodiment is shown. As disclosed above, the plasmon antenna 106 is positioned within the waveguide 103 at the intersection of sampling channel 104 and the coupling channel 105. In the disclosed embodiment, the sampling channel 104 has a width ranging between 10-15 nanometers and the coupling channel has a width ranging between 27-42 nanometers.

As shown in FIG. 2A, the plasmon antenna 106 is comprised of nanostructures 201, 202 positioned on each side of and coupled to a nanoaperture 203.

The nanoaperture 203 is affixed to the outer surface of the sampling channel 104. It is purposely positioned within the sampling channel 104 as to be in-line with any molecules suspended within a fluid pumped though the sampling channel 104. The nanoaperture 203 may be composed of any coinage metal capable of surface plasmon resonance including gold and silver. The nanoaperture 203 may also be composed of any material not capable of surface plasmon resonance.

The nanostructures 201, 202 are affixed to the outer surface of the coupling channel 105 and are positioned on opposing sides of the nanoaperture 203, a portion of each nanostructure 201, 202 in direct contact with an outer surface of the nanoaperture 203.

Alternatively, the nanostructures 201, 202 may be embedded within an additional layer of silicone dioxide (SᵢO₂) grown over the waveguide 103. In this alternative configuration, the nanostructures 201, 202 are position on the silicone nitrate (Si₃N₄) surface of the waveguide 103 and an additional layer of silicon dioxide (SᵢO₂) is grown around the nanostructures 202, 202. The nanostructures 202, 202 are positioned and configured to form a gap which will function as a plasmon antenna. Once the additional layer of silicon dioxide (SᵢO₂) has been fully grown, the sampling channel 104 is etched into this additional layer running through the gap in between the embedded nanostructures 201, 202. In this alternate configuration, the sampling channel 104 alone is etched into the additional layer without an intersecting coupling channel 105. As discussed above, the nanoaperture 203 is affixed to the outer surface of the sampling channel 104 and is in direct contact with the nanostructures 201, 202 embedded within the additional layer of silicon dioxide (SᵢO₂) and opposing sides of the nanoaperture 203. The nanoaperture 203 is specifically positioned as to be in-line with any molecules suspended within a medium pumped though the sampling channel 104. In this alternative configuration, the embedded nanostructures 201, 202 are more directly coupled to the waveguide 103 than when affixed directly to the surface of a channel etched into the waveguide 103.

The nanostructures 201, 202 are composed of gold, silver, or any other coinage metals capable of surface plasmon resonance. Alternatively, the nanostructures may be composed of multiple layered metals, each metal capable of plasmon resonance.

In the disclosed embodiment, the nanostructures 201, 202 are shown as triangular shaped and in a bowtie configuration bordering and in direct contact with the nanoaperture 203. This bowtie configuration helps to tightly confines the optical field in the gap where the nanoaperture 203 is positioned and thereby creates a plasmon field apex within the area surrounding the nanoaperture 203. In the disclosed embodiment, the length of each leg of the nanostructures 201, 202 ranges between 25-40 nanometers.

While the disclosed embodiment shows triangular shaped nanostructures 201, 202, nanostructures of other shapes may be implemented to achieve a localized plasmon field apex surrounding the nanoaperture 203. These other shapes include rods, spheres, trapezoids, or any other shapes known to one of ordinary skill in the art.

In FIG. 2B, an inline view of the plasmon antenna implemented within the portable single-molecule bio-sensing device according to a disclosed embodiment is shown. As shown in FIG. 2B, the nanoaperture 203 includes one or more apertures 204 in line with the sampling channel 104 through which all fluid samples 205 must pass.

The disclosed embodiment shows a rectangular block shaped nanoaperture 203. However, the nanoaperture 203 may have any shape encompassing one or more apertures 204 through which the fluid samples 205 must pass through, including spherical and ring shaped structures.

The nanoaperture 203 is attached to the outer surface of the sampling channel 104 without penetrating the outer surface. In the disclosed embodiment, the width of the nanoaperture ranges between 5-10 nanometers, thereby fitting fully within and encompassing a substantial portion of the sampling channel 104.

The nanoaperture 203 restrict the movement of molecules through the plasmon antenna 106. The molecules are pulled and focused through the one or more apertures 204, the aperture functioning as a gateway through the gap between the nanostructures 201, 202 where the plasmon field apex is located. This restriction of molecule movement through the nanoaperture 203 creates a single point of measurement that helps increase the accuracy and reproducibility data generated by the plasmon antenna 106.

The nanoaperture 203 also allows for easier functionalization of the plasmon antenna 106 as nucleotide bases, chemical, receptors or other functional groups may be attached directly to the nanoaperture 203 rather than to the nanostructures 201, 202 or the surface of the waveguide 103.

In FIG. 2C, an inline view of an alternate nanoaperture shape implemented within the portable single-molecule bio-sensing device according to a disclosed embodiment is shown. As shown in FIG. 2C, the nanoaperture 203 is shaped to provide for larger particles such as viruses or even cells and their components. The nanoaperture 203 is shaped to perform as a cap over the sampling channel 104 with a tunnel like aperture 204 through which fluid samples 205 is pumped. The larger particles rub up against and are squeezed through the aperture 204 which is the plasmon filed apex. The resulting prolonged interaction between the larger particles to be identified and the plasmon field, inclusive of particles rubbing up against the nanoaperture 203, results in a larger and more easily detectable refractive index change without functionalizing the nanoaperture 203.

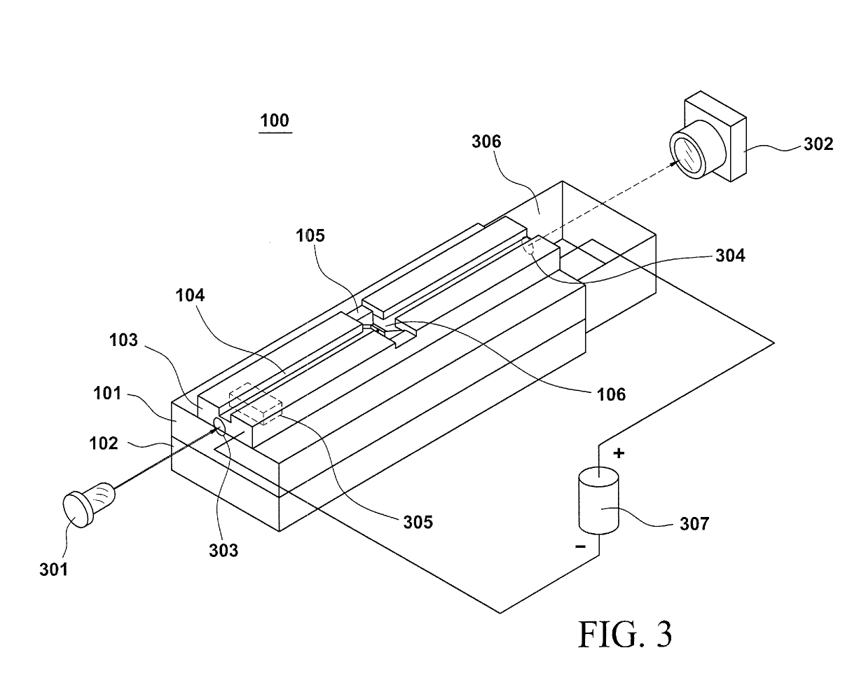

In FIG. 3, a block diagram showing a light source unit, a photodetector unit, a micropump, and a power source as implemented in conjunction with the portable single-molecule bio-sensing device according to the disclosed embodiment is shown.

As shown in FIG. 3, the light source unit 301 supplies light at one end 303 of the waveguide 103. A light propagating through the waveguide 103 causes coupling between the evanescent waves at the surface of the planar waveguide to the nanoparticles comprising the nanostructures 201, 202. At a resonant wavelength, a plasmon polariton is excited at the surfaces of the nanostructures 201, 202. The resonant wavelength is calculated based on the physical structural characteristics of the nanostructures 201, 202 and the refractive index of the sample fluid, which for water is a constant 1.33. In operation, the light source unit 301 output wavelength may be varied until a dip in intensity occurs at a certain wavelength, this certain wavelength being the resonant wavelength for the specific nanostructures 201, 202.

Moreover, if the nanoaperture 203 is itself composed of a material known to excite plasmons, the resonant wavelength may need to be shifted based on the presence of the nanoaperture 203 in the gap between the nanostructures 201, 202. The resulting surface plasmon resonance is dependent on the refractive index of the environment surrounding the plasmon antenna as well as the resonant wavelength used to excite the plasmon polariton. The presence of a molecule within the plasmon antenna will alter the refractive index of the surrounding environment and create a detectable shift in the plasmon resonance wavelength specific to each molecule.

In the disclosed embodiment, a polarized and high intensity light source is used to initiate surface plasmon polariton. However, incoherent light sources may also be used as long as they include those wavelengths needed for plasmon field excitation. As an example, a light wavelength range between 6-1100 nanometers is suitable.

The light source unit 301 may be integrated within the device 100 or may be implemented externally using a single-mode fiber optic cable to deliver light from the light source unit 301 to a first end 303 of the waveguide 103.

The photodetector unit 302 receives light from a second end 304 of the waveguide 103. The photodetector unit 302 is designed to detect changes in light originating from the light source unit 301 due to molecule translocation through the plasmon antenna 106. The photodetector unit 302 may be an avalanche photodetector, a mini spectrometer, or any other type known to one of ordinary skill in the art.

The photodetector unit 302 may be integrated within the device 100 or may be implemented externally using a multi-mode fiber optic cable to deliver light from the waveguide 103 to the photodetector unit 302. A filter or array of filters may also be implemented in line with the light exiting the second end 304 of the waveguide 103, the filters correlating to specific wavelength shifts that identify specific nucleotide bases.

The plasmon field localized in between the nanostructures 201, 202 of the plasmon antenna 106 is highly sensitive to changes in the refractive index of the environment surrounding the plasmon antenna 106. Changes in the refractive index of the surrounding environment can be the result of its interaction with molecules passing through that surrounding environment. These changes in refractive index can be conveyed as shifts in the resonant wavelength and changes in the intensity of the light originating from the light source unit 301 after having interacted with plasmon antenna 106. Specifically, the interaction of molecules with the plasmon field emanating from the plasmon antenna causes changes to the frequency of the plasmon field localized and concentrated in the gap between the nanostructures. These changes in the frequency of the plasmon field create a shift in the resonant wavelength and in the intensity of the light traveling through the waveguide 103 and measured by the photodetector unit 302. Specific wavelength and intensity shifts may be correlate to specific nucleotide bases.

These shifts in frequency and intensity are measured directly in order to discriminate between different nucleotide bases or other molecular components of biomolecules. A specific molecular component may be associate with a specific shift in frequency, a specific shift in intensity, or a combination of both for greater accuracy.

This method of measurement provides for a much smaller and simpler method of molecular detection than existing light based detection methods such as surface enhanced Raman spectroscopy. This reduction in scale and complexity allows for chip based implementations. This measurement method also provides for far greater detection speeds than existing electric current measurement methods.

A micropump 305 is implemented to pump fluids and any molecules suspended therein through the plasmon antenna 106. The micropump pump 305 is electrically controlled and may be either integrated within the waveguide 103 during fabrication or it may be external to the waveguide. In either configuration, the micropump 305 is coupled to the sampling channel 104 in a manner that provides for controlling the flow of fluid along the surfaces of the sampling channel 104 and the coupling channel 105. The micropump 305 may further implement filters that filter a fluid sample to remove certain molecules and waste particles prior to passing that fluid sample through the plasmon antenna 106.

A power source 307 is connected to each end of the waveguide 103 such as to provide an electric field across the sampling channel 104. This electric field helps straighten and align molecules travelling through the sampling channel 104. This straightening and alignment provides for a more efficient translocation of the molecules through the plasmon antenna 106.

A chamber 306 is located at an end 304 of the device 100 to collect fluid that has traveled through and exists the sampling channel 104.

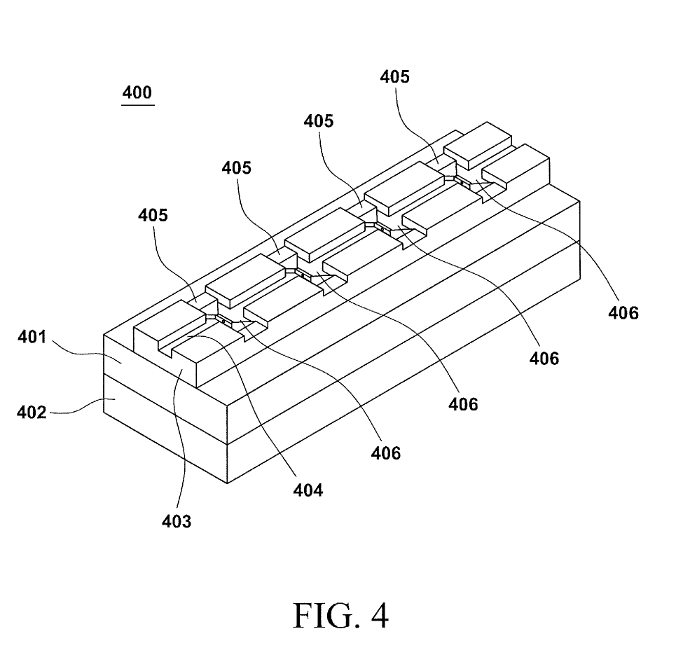

In another embodiment, a plurality of plasmons antennas may be provided on a single portable single-molecule bio-sensing device. In FIG. 4, a diagram of a portable single-molecule bio-sensing device according to another embodiment is shown. Similar to the embodiment shown in FIG. 1, the portable single-molecule bio-sensing device 400 in this other embodiment includes a waveguide 403, a top layer substrate 401, a lower layer substrate 402 and a sampling channel 404. As shown in FIG. 4, in this embodiment, the single-molecule bio-sensing device 400 includes a plurality of coupling channels 405 and multiple plasmon antennas 406 positioned at the intersections of each of the plurality of coupling channels 405 and the sampling channel 404. Each plasmon antenna 406 is comprised of the same component as the embodiment shown in FIGS. 2A and 2B. However, the nanoaperture in each plasmons antenna 406 may be functionalized differently to detect and react to different types of molecules.

In yet another embodiment, the molecules to be detected are translocated through a plasmon antenna via an air stream rather a fluid. In this embodiment, the sampling channel 104 and the coupling channel 104 are designed as an air tight enclosure that provides for the chamber through which an air sample may be taken in incremental amounts.

The micropump 305 is implemented to pump a stream of air and any molecules suspended therein through the plasmon antenna 106. The micropump pump 305 is electrically controlled and may be either integrated within the waveguide 103 during fabrication or it may be external to the waveguide. In either configuration, the micropump 305 is coupled to the sampling channel 104 in a manner that provides for controlling the flow of air within the sampling channel 104 and the coupling channel 105. The micropump 305 may further implement filters that filter and air sample to remove certain molecules and waste particles prior to passing that air sample through the plasmon antenna 106.

What is claimed:

1. A portable single-molecule bio-sensing device, comprising:
a substrate;
a waveguide positioned on an upper surface of the substrate;
a sampling channel edged into the waveguide, the sampling channel running a longitudinal length of the waveguide;
a coupling channel edged into the waveguide, the coupling channel running perpendicular to the sampling channel;
a pair of nanostructures secured to an outer surface of the coupling channel on opposite sides of the sampling channel, the nanostructures configured to form a gap that functions as a plasmon antenna; and
a nanoaperture affixed to an outer surface of the sampling channel within the gap, an outer surface of the nanoaperture in direct contact with each of the pair of nanostructures and the nanoaperture including at least one aperture extending the full length of nanoaperture and in-line with the sampling channel.

2. The portable single-molecule bio-sensing device of claim 1, wherein the substrate is composed of an upper layer of silicon dioxide (SiO₂) over a lower layer of silicon (Si), the upper layer having a thickness less than or equal to 5 mm.

3. The portable single-molecule bio-sensing device of claim 1, wherein the waveguide is composed of silicon nitrate (Si₃N₄).

4. The portable single-molecule bio-sensing device of claim 1, wherein the waveguide is either a ridge type waveguide, a slot type waveguide, or a plasmonic type waveguide.

5. The portable single-molecule bio-sensing device of claim 1, wherein the waveguide has a width ranging between 400-900 nanometers and a height ranging between 200-300 nanometers.

6. The portable single-molecule bio-sensing device of claim 1, further comprising an adhesion layer over the outer surfaces of the sampling channel and the coupling channel, the adhesion layer composed of either a titanium (Ti) or chromium (Cr) based material and having a thickness ranging between 1-3 nanometers.

7. The portable single-molecule bio-sensing device of claim 1, further comprising a photodetector receiving light from the waveguide, the photodetectors detecting shifts in frequency and intensity resulting from the translocation of a molecule through the plasmon antenna.

8. The portable single-molecule bio-sensing device of claim 1, further comprising a micropump attached to the waveguide, the micropump enabling and controlling a translocation of medium containing samples through the plasmon antenna.

9. The portable single-molecule bio-sensing device of claim 8, wherein the medium is deionized water with a constant refractive index of 1.33.

10. The portable single-molecule bio-sensing device of claim 8, wherein the medium is air with a constant refractive index of 1.00.

11. The portable single-molecule bio-sensing device of claim 1, wherein the nanostructures are triangles in a bowtie configuration.

12. The portable single-molecule bio-sensing device of claim 1, wherein width of the nanoaperture ranges between of 5-10 nanometers.

13. The portable single-molecule bio-sensing device of claim 1, wherein the nanoaperture and the nanostructures are both made of any coinage metal know to support surface plasmon resonance.

14. The portable single-molecule bio-sensing device of claim 1, wherein the nanoaperture is made of a thin layer of graphene.

15. The portable single-molecule bio-sensing device of claim 8, further comprising filters at the fiber-optic any of the connections between the waveguide and the photodetector, the filters filtering out a set of wavelengths that are characteristic of bases and/or of a unique sequence of a nucleic acid or molecule to be detected.

16. The portable single-molecule bio-sensing device of claim 1, wherein the nanoaperture is functionalized.

17. The portable single-molecule bio-sensing device of claim 7, further comprising a computing means for analyzing intensity and frequency data provided by the photodetector, the analysis consisting of matching shifts in frequency and intensity detected by the photodetector to previously identified and stored sequences of shifts in frequency and intensity correlating to specific molecular bases.

18. A method of bio-sensing a single-molecule, the method comprising:
energizing a plasmon field on the surface of a waveguide;
introducing a medium containing a nucleic acid, protein, molecules and viral/cell components to be sampled into a sampling channel etched into the waveguide;
translocating the medium though a nanoaperture in direct contact with surrounding nanostructures forming a plasmon antenna;
detecting shifts in light intensity and frequency produced by the interaction of the nucleic acid or protein with the plasmon field as it is translocated through the plasmon antenna; and
identifying the translocated nucleic acid or protein based on the detected shifts in light intensity and frequency.

19. A portable single-molecule bio-sensing device, comprising:
a substrate;
a waveguide positioned on an upper surface of the substrate;
an additional layer of silicone dioxide (SiO₂) grown on an upper surface the waveguide;
a pair of nanostructures embedded within the additional layer, the pair nanostructures configured to form a gap that functions as a plasmon antenna;
a sampling channel edged into the additional layer, the sampling channel running a longitudinal length of the additional layer and positioned within the gap that functions as the plasmon antenna; and
a nanoaperture affixed to the outer surface of the sampling channel, the nanoaperture positioned to be in direct contact with each of the pair of nanostructures and the nanoaperture including at least one aperture extending the full length of nanoaperture and in-line with the sampling channel.
